(19) United States
(12) Patent Application Publication
(10) Pub. No.: US 2022/0167916 A1
(43) Pub. Date: Jun. 2, 2022

(54) INTELLIGENT DIET, SLEEP AND STRESS MANAGEMENT

(71) Applicant: UNIVERSITY OF NORTH TEXAS, Dallas, TX (US)

(72) Inventors: Saraju P. MOHANTY, Dallas, TX (US); Elias KOUGIANOS, Dallas, TX (US); Laavanya RACHAKONDA, Dallas, TX (US)

(21) Appl. No.: 17/540,076

(22) Filed: Dec. 1, 2021

Related U.S. Application Data
(60) Provisional application No. 63/134,820, filed on Jan. 7, 2021.
(60) Provisional application No. 63/120,635, filed on Dec. 2, 2020.

(51) Int. Cl. A61B 5/00; A61B 5/11; A61B 5/16; G16H 20/30; G16H 20/70; G16H 20/60

(52) U.S. Cl. CPC A61B 5/6823; A61B 5/4806; A61B 5/1116; G16H 20/60; A61B 5/165; G16H 20/30; G16H 20/70; A61B 5/7264

(57) ABSTRACT

A wearable device including multiple sensors, a memory, a processor coupled to the memory and the multiple processors, and executable code stored in the memory. When executed by the processor, the executable code causes the processor to receive data from at least some of the multiple sensors, the data indicating physical activity of a wearer of the wearable device, food consumption of the wearer, and sleeping habits of the wearer and determine an estimated stress level of the wearer based on the physical activity of the wearer, the food consumption of the wearer, and the sleeping habits of the wearer.

### CROSS REFERENCE TO RELATED APPLICATIONS

This application claims the benefit of 1) U.S. Provisional Application No. 63/120,635, filed on Dec. 2, 2020, and entitled “MY-PAL: AN INTELLIGENT DEVICE FOR AUTOMATIC DIET, SLEEP AND STRESS MANAGEMENT,” and 2) U.S. Provisional Application No. 63/134,820, filed on Jan. 7, 2021, and entitled “INTELLIGENT DIET, SLEEP AND STRESS MANAGEMENT,” both of which are incorporated herein by reference in their entirety for all purposes.

### STATEMENT REGARDING GOVERNMENTALLY SPONSORED RESEARCH OR DEVELOPMENT

Not applicable.

### BACKGROUND

Various attempts have been made to help users monitor their daily food intake, for example, using various applications and electronic devices. For example, systems that consider current food intake and predict future food intake as necessary to maintain a healthy diet and/or weight have been proposed. However, these applications and devices often fail to address stress and/or consider the relationship between food intake and stress.

Further, many systems rely upon manual user inputs with respect to the food items and information about stress and food already consumed, meaning that these systems are often unreliable. For example, few visual approaches exist that use an external camera to capture and process (e.g., determine) food volume and estimate food weight and, moreover, these approaches may fail to implement an internet of things (IoT) approach. Many approaches to monitoring food intake have thus proven inaccurate and ineffective. Likewise, such approaches may fail to address the relationship between stress and food consumption.

As such, new and improved methods for monitoring food intake and correlating stress levels are needed.

### SUMMARY

In some embodiments, a wearable device comprises a plurality of sensors, a memory, and a processor. The processor is coupled to the memory and the plurality of sensors, and executable code is stored in the memory that when executed by the processor, causes the processor to: receive data from at least some of the plurality of sensors, the data indicating physical activity of a wearer of the wearable device, food consumption of the wearer, and sleeping habits of the wearer; and determine an estimated stress level of the wearer based on the physical activity of the wearer, the food consumption of the wearer, and the sleeping habits of the wearer.

In some embodiments, a method for stress control comprises capturing or receiving data from at least some of the multiple sensors, the data indicating physical activity of a wearer of the wearable device, food consumption of the wearer, and sleeping habits of the wearer, determining an estimated stress level of the wearer based on the physical activity of the wearer, the food consumption of the wearer, and the sleeping habits of the wearer, and automatically determining and recommending a stress control remedy to the wearer based on the determined estimated stress level and at least one of the physical activity of the wearer, the food consumption of the wearer, or the sleeping habits of the wearer.

These and other features will be more clearly understood from the following detailed description taken in conjunction with the accompanying claims.

### BRIEF DESCRIPTION OF THE DRAWINGS

For a more complete understanding of the present disclosure and the advantages thereof, reference is now made to the following brief description, taken in connection with the accompanying drawings and detailed description, wherein like reference numerals represent like parts.

FIG. 1 illustrates a schematic representation of an embodiment of a proposed system for stress management.

FIG. 2 illustrates and exemplary schematic architecture of the My-PAL platform.

FIG. 3 illustrates an exemplary schematic high-level architecture of an embodiment of the My-PAL wearable.

FIG. 4 illustrates another exemplary schematic architecture of an embodiment of the My-PAL.

FIG. 5 illustrates an exemplary flowchart of a process of My-PAL according to an embodiment.

FIG. 6 illustrates an exemplary schematic of a neural network or Machine Leaning Model architecture that may be used for the process of automatic stress detection and prediction according to some embodiments.

FIG. 7 illustrates an exemplary data flow from the My-PAL wearable to the My-PAL user interface according to some embodiments.

FIG. 8 illustrates the stress behavior analyses from the wearable to the user interface according to some embodiments.

FIG. 9 illustrates an exemplary process of automatic processing in My-PAL for image detection, object classification, and calorie quantification according to some embodiments.

FIG. 10 illustrates an exemplary methodology for automatic stress detection according to some embodiments.

FIG. 11 illustrates an exemplary stress prediction system for My-PAL according to some embodiments.

FIG. 12 illustrates a number of different approaches for stress control according to long term and short-term control mechanisms according to some embodiments.

### DESCRIPTION

Psychological stress affects the physiological parameters of a person. Prolonged exposure to stress can have detrimental effects which might require expensive treatments. To self-manage this important health problem in the framework of smart healthcare, a deep-learning based system (My-PAL) is proposed. Stress is broadly categorized into distress, which is negative stress, and eustress, a positive stress. Stress can be further classified in three different categories: acute stress, episodic acute stress and chronic stress. Acute stress is short-term while episodic acute stress is the repetition in the frequency of occurrence of acute stress.

Chronic stress is the result of prolonged exposure to stressors. Increase in stress levels can push a person to complex mental illnesses such as borderline personality disorder (BPD), which causes dangerous mood swings, changes in behavioral patterns, eating disorders and may provoke the stressed person to take unhealthy decisions. Not knowing when to stop eating or how much food is too much can lead to many health issues. Chronic stress, uncontrolled or unmonitored food consumption, not having adequate sleep and obesity are intricately connected, even involving certain neurological adaptations. Being able to live happily and peacefully should not be as difficult as it is in today's society.

Having emotions and reacting to events is good if there is a healthy stress response. When a person does not know how to cope with stress, technology may be able to provide a solution. Often, people don't realize that they are under stress until the very last moment, meaning that people may respond to unrecognized stresses in unhealthy ways. In order to avoid these unhealthy responses, a stress control system is proposed in My-PAL. Using My-PAL, the physical activity of the user, the food consumption rate, and the sleep data may be analyzed, and the user may be notified with the stress control mechanisms provided through a mobile application interface or other notification mechanism. The collected data may be transmitted to and stored in the cloud, which may allow for real-time monitoring of a person's stress levels and diet and sleep habits, thereby reducing the risk of death and expensive treatments.

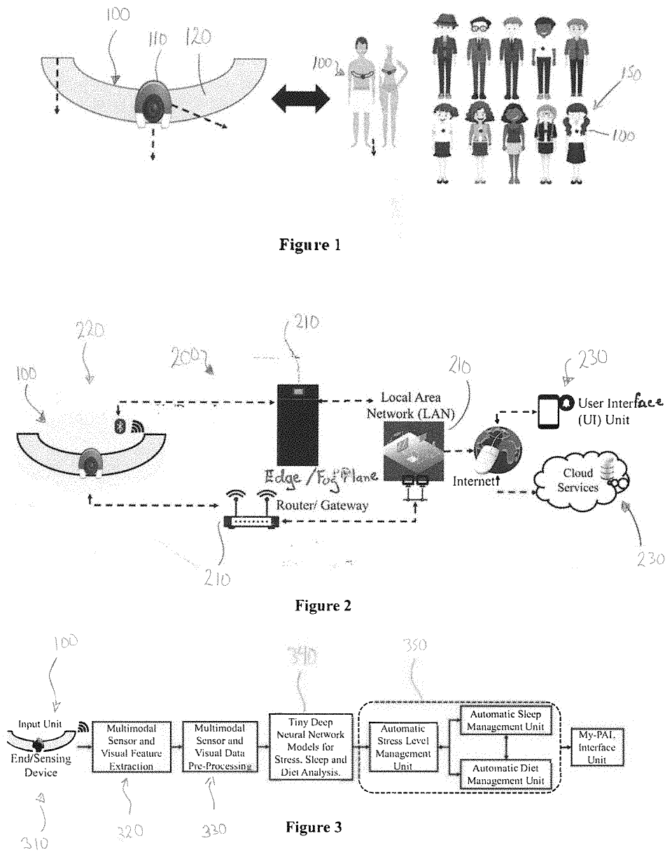

An example of a proposed system for stress management that considers the physical activity, diet and/or sleep habits of the person is represented in FIG. 1. FIG. 1 demonstrates an embodiment of a user device 100, for example, a My-PAL used as a marketable device. In the embodiment of FIG. 1, the user device 100 generally includes an internet-of-things (IoT)-capable component (referred to herein as an IoT component 110) and a wearable component 120. In some embodiments, the IoT component 110 may comprise a device having one or more sensors, processors, memory, and communications components (e.g., transmitters, receivers, transceivers, etc.) configured for communication with one or more other devices, as will be disclosed herein. In various embodiments, the sensors may be configured for the collection of various data, as will also be disclosed herein, for example, comprising camera(s), motion sensor(s), orientation/position sensor, accelerometer(s), the like, and combinations thereof. Also in the embodiment of FIG. 1, the IoT component 110 may be worn by a wearer or user 150, for example, by engagement of the IoT component 110 with the wearable component 120, such as via a magnet or a suitable mechanical engagement. In the embodiment of FIG. 1, the wearable component 120 is illustrated as a band which may be on or as a part of various outfits suitable for the daily life, for example, about the torso of the user 150.

In some embodiments, the user device 100 (e.g., My-PAL) may be configured to implement various additional features, for example, food analysis and/or processing and physical activity monitoring. The stress management techniques have been increased and the wearable may have the capability to connect to other IoT devices, for example, to control the sudden stress level fluctuations. Additionally or alternatively, in some embodiments the user device 100 (e.g., My-PAL) may be configured to analyze and/or manage diet and sleep. For example, a stress control remedy may be implemented dependent upon stress level fluctuations during sleep may include a sleep management action, which can control the room ambiance, room temperature, a connection to adjust the bed according to the human body posture and regulate the mattress temperature according to the stress level, a connection to the essential oil dispenser to regulate the stress levels, a connection to smart mobile phone or personal assistant device to play soothing sounds when the person is under stress.

The user device 100 (e.g., My-PAL) may be effective to remedy, mitigate, or otherwise address various problems or shortcomings, such any one or more of the following:
Problem of automatic analysis of food intake monitoring without having human input.
Problem of automatically detecting the type of food consumed without having human input.
Problem of automatic quantification of calorie value of food intake without human input.
Problem of not having an accurate stress detection methodology.
Problem of not considering all the possible factors that could affect the fluctuations in stress.
Problem of not having the understanding that human stress has a direct/indirect relationship to the lifestyle of the user.
Problem of not having an IoT-edge computing mechanism with which the performance, accuracy and stabilization of the system can be maintained.
Problem of not considering the relationship between sleeping habits with the stress level fluctuations.
Problem of not considering the side effects of overeating and its relationship to stress.
Problem of not having the mechanisms to analyze stress during the sleeping periods.
Problem of lack of electronics that can provide remedies for stress fluctuations for fast relief.
Problem of not having a unified system that considers the human behaviors and chores to analyze stress and suggest control remedies to the user.

For example, to remedy, mitigate, or otherwise address at least some of the above problems or shortcomings, the user device 100 (e.g., My-PAL) may include, or implement, any one or more of the following features:
The user device 100 may perform multimodal, accurate human stress detection considering the lifestyle of a user by taking into consideration factors such as physical activity performed, food consumed, and/or the sleeping habit style.
Appropriate automated stress control remedies may be determined and recommended for a normalized stress pattern.
A method that includes automatic food logging using object classification from images with no user input, such as at a time of food intake or preparation.
A method that includes automatic calorie quantification without manual entry of food intake for detecting an accurate stress level.
A method that includes considering leftover food and the unconsumed calories for stress detection.
A method that includes considering the physiological parameter changes during sleep of a person for accurate and rapid stress detection.
A method that includes automatically monitoring the daily activities and/or chores of the person to analyze stress fluctuations.
A method that includes automatically monitoring and considers workouts, number of steps taken, and/or number of calories burnt to analyze stress levels.
A method that includes considering leftover calories or the number of calories that can be consumed and suggesting diet options for effective and/or healthy lifestyle.
A method that includes automatically providing or suggesting a sleep management schedule or a plan for the user to make the most of the process in relationship with stress.
A method that includes suggesting diet management concepts by having both diet control and monitoring for a healthy life in relationship with stress.
A method that includes providing workout plans or yoga plans based on the user's stress fluctuations.
A wearable device with connected properties that may enable it to enhance the sleep, control the food consumption and regulate the physical activity of a user to maintain a stable stress level of the user.
A method that includes addressing acute stress by providing automated short-term remedies of stress.
A method that includes addressing chronic stress by providing automated long-term remedies of stress.
A wearable that may use location services to provide accurate long-term stress remedies when a user is in need.
An interface that allows the users to access the information and thereby educate themselves about their stress fluctuations.
A method that includes stress predictions, thereby providing users with a time period allowing the user to take control over stress.
An Internet-of-Medical-Things (IoMT) based healthcare Cyber-Physical System (H-CPS) framework that has multiple (e.g., 4) different approaches for stress control.
A method that includes monitoring the physiological signals including the sleep latency or the food consumption for the following day stress predictions.
A method that includes educating the users on the difference between living a life with controllable normalized stress and uncontrollable prolonged stress.
An IoMT based H-CPS framework for unified and/or automatic diet, sleep and stress management.

Referring to FIG. 2, an example architecture of a platform 200 in which the user device 100 can operate is shown. For example, the platform 200 illustrates the user device 100 (My-PAL) in an Internet-of-Medical-Things (IoMT)-based Healthcare Cyber-Physical System (H-CPS) Framework. As shown in the embodiment of FIG. 2, the processing and/or operation of the user device 100 (My-PAL) can be performed in/on edge-level devices 210 (e.g., a router, a gateway, etc.), in/on low-performance computing environments 220 such as within sensors and/or single board computers, and/or in/on high-computing devices 230 such as an In-Cloud units (e.g., a server), each of which can be connected using a wired and/or wireless connection such as a network.

Referring to FIG. 3, an example of a high-level architecture of the user device 100 (e.g., My-PAL) is illustrated including Proposed Multimodal Computing Units in the user device 100 (e.g., My-PAL). As shown in FIG. 3, the user device (e.g., My-PAL) may comprise a number of sensors such as movement sensor(s) (e.g., an accelerometer, gyroscope, vibration sensor, etc.), positioning or location sensor(s) (e.g., GPS, local area network location determination, etc.), acoustic sensor(s), and/or visual sensor(s) (e.g., camera(s), infrared detectors, etc.). Further, the user device 100 may be IoT-capable or friendly and may be capable of connection to other devices, such as smart personal assistance devices, environmental device, and the like to regulate stress levels for stress control mechanisms. In some aspect, the user device 100 (My-PAL) can comprise suitable communication devices or components to enable various communication schemes such as WiFi, Bluetooth, near-field communication, cellular connections such as data connections, and the like, which can enable network connections and/or IOT connections to other devices.

In some embodiments, signal data and/or image data may be obtained 310 from the user device 100, for example, via the IoT component 110. The data can comprise any of the outputs of the sensors described herein. In some aspects, food intake data can comprise an age of the user, a gender of the user, a total calorie count, a time since a last meal, a type of food consumed, an amount of food consumed, an amount of food not consumed (e.g., left without consuming), a calorie count of food not consumed, an amount of time to consume the food, or any other suitable sensor data obtained from the sensors. In some aspects, physical activity data can comprise movement data, temperature readings, accelerometer readings (e.g., amplitude of movements, speed, etc.), skin conductance rate, heart rate, respiration rate, pupil movement rate, forehead frown reading, gyroscope reading, total number of steps, total calories burned, time of activity, resting time without movement, or the like. In some aspects, sleep tracking data can comprise slow-wave monitoring, eye movement rate, heart rate, respiration rate, blood oxygen level, body position readings, chest and abdominal movement (e.g., breathing rate), limb movement, snoring rate, time spent sleeping, sleep latency, and the like.

Various features may be extracted 320 from the signal data and/or image data and the signal data and/or image data may be processed 330, for example, prior to further analysis. For example, in various embodiments, the IoT component 110 may be a heart-rate sensor, a respiration-rate sensor, or an accelerometer, any of which may produce raw data when in use, (for example, in the context of use by a human being). For example, a pulse sensor may be used here for heart-rate monitoring. The pulse sensor may have two surfaces; a first surface with a light emitting diode and an ambient sensor diode, and the second surface with the circuitry for noise cancellation and amplification. The first surface having the light-emitting diode (LED) may be placed directly on the skin, such as on an earlobe or fingertip and, when placed, the LED starts to emit light. As blood flows through the veins, the ambient light sensor will receive an increasing light with respect to the blood flow. Thus, change in the light received over time may be used to determine the pulse rate as a feature from the raw data. Heart-rate (e.g., beats per minute) may be determined and vary accordingly with the blood flow.

In some embodiments, and as will be disclosed herein, the data (e.g., one or more extracted features) may be analyzed via a machine learning model 340 (e.g., deep machine learning models) that analyze the data, for example, sleeping habits of the person for the last night's sleep, the food consumed throughout the day and/or the physical activity performed by the user. Based upon the analysis of this information, fluctuations in stress may be identified and/or determined. In various embodiments, the machine learning model 340 produce various outputs corresponding to the various inputs that are fed into the machine learning model 340. For example, for the sensor data, a Comma Separated Value (CSV) file may be fed to the machine learning model 340 as the input; for image analysis, images may be converted to an Extensible Markup Language (XLM) format and fed to the machine learning model 340. In some embodiments, the outputs corresponding to such inputs may be characterized as dictionaries, for example, lists with predictions before and after training the machine learning model 340 and images with bounding boxes along with the confidence percentage of the machine learning model 340, respectively. In some embodiments, the one or more stress relief mechanisms may be implemented and/or provided 350, such as via the mobile application or other graphical interface. In some embodiments, the analysis of activity with respect to food consumption, sleep tracking and physical activity tracking may be presented 360 to the user using the mobile application or other graphical interface.

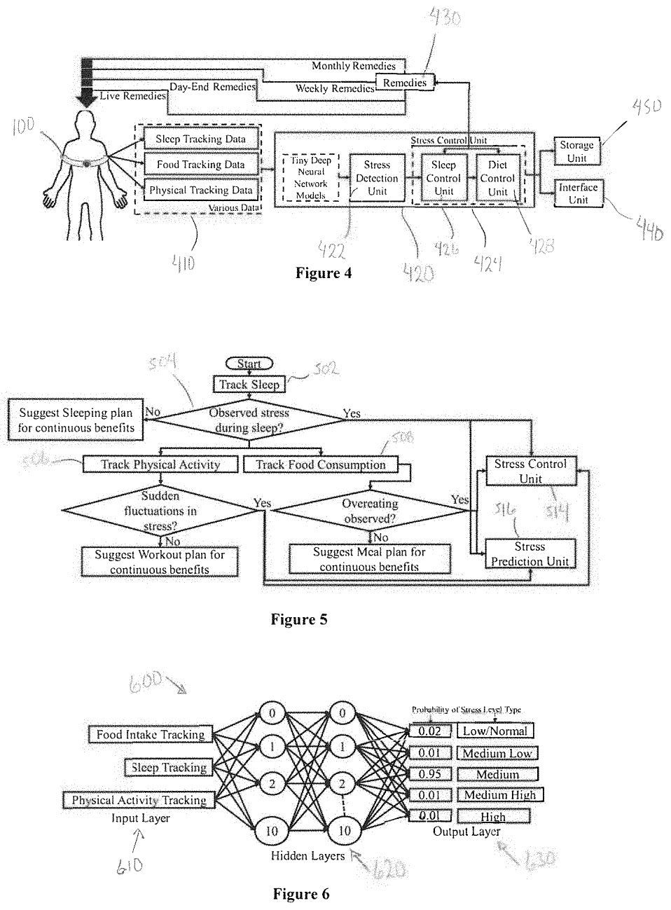

Referring to FIG. 4, an example of an architecture for the operation of the user device (e.g., My-PAL) is represented. As shown in FIG. 4, data 410 from a user device 100 and/or one or more features extracted from the data may be monitored and sent to the neural network models 420, for example, to implement various processes such as via a stress detection unit 422. The data 410 can comprise any data obtained from the sensors and/or the features as described herein. Additionally, the neural network models 420 may implement various processes to control stresses, for example, via a stress control unit 424 which may include one or more subunits, such as a sleep control unit 426 and a diet control unit 428. The neural network models 420 can provide one or more outputs indicative of a stress of the user. In response, one or more remedies 430 may be provided as a feedback to the user, which can in some aspects be based on the output of the neural network models 420. For example, one or more remedies may be provided to the user via the interface unit 440 (e.g., a mobile application interface). The remedies 430 may be a combination of both automatic suggestions and non-automatic suggestions. Various data and/or analysis, such as produced via the neural network models 420, may also be provided to the user such as via the interface unit 440. Also, in some embodiments, various data and/or analysis may be stored such as in a storage unit 450.

Referring to FIG. 5, an example flowchart (e.g., decision-tree) of a process of using the user device 100 (My-PAL) is represented. In some embodiments, as an example, decisions relating to stress fluctuations may be made using a range comparison process. The working process may start from the person going to bed (step 502), drifting to sleep and waking up. The changes in physiological signal parameters that vary during sleep may be monitored and the stress during sleep may be determined (step 504). For example, any of the models and processes described herein can be used to determine the stress during sleep such as using physiological parameters and/or features extracted from the physiological parameters as inputs into a machine learning model such as a neural network to provide an output indicative of the stress during sleep. Later, when the person is awake, the process of physical activity tracking (step 506) and/or food consumption (step 508) may be performed until the person goes to bed again, thus, completing a cycle. Any of the models and processes described herein can be used to determine the stress during the day such as using the physical activity data, food consumption data, and/or features extracted from the physical activity data and/or food consumption data as inputs into a respective machine learning model such as a neural network to provide an output indicative of the stress during the day. Depending upon the results of physical activity tracking and/or food consumption, recommendations (steps 510 and 512) may be provided to the user and/or measures to mitigate and/or predict stress may be implemented (steps 514 and 516).

In various embodiments, a neural network and/or machine leaning model architecture may be represented as including layers and neurons that may be used for the process of automatic stress detection and prediction. For example, in the embodiment of FIG. 6, the machine learning model is as shown, for example, a proposed tiny deep neural network (DNN) model for implementation of the user device 100 (My-PAL). In the embodiment of FIG. 6, a Fully Connected Neural Network (FCNN) model 600 includes a linear stack of a single input layer 610, two hidden layers 620, and a single output layer 630. The FCNN model 600 is illustrated as including ten (10) neurons implemented so as to establish the relationship between the physiological parameters (e.g., the inputs) and stress levels (e.g., the outputs). The outputs can be presented in a variety of format. In some aspects, the output of the model may provide a probability of the stress level falling within various pre-defined stress levels. The stress level with the highest probability can be selected as the stress level of the user. While five stress levels are shown in FIG. 6, any number of stress levels (e.g., buckets of stress levels) can be defined, and the model can be trained to identify the probability for each stress level. In general, a large number of pre-defined stress levels may require a larger training data set, but there is no limit to the number of stress levels that can be used with the model.

An example of a data training methodology of the tiny DNN models suitable for use with the user device 100 (My-PAL) can begin with setting an epoch value and iterating each stress epoch. This epoch defines the number of times the dataset loops. Inside each repetition, each example can iterate from the training dataset (e.g. a labeled data set) by correlating its input features (e.g., the physiological parameters) and the output labels (e.g., the stress levels). Using these features and training dataset, inferences or predictions are made. Actual stress level outputs can then be compared with the stress predictions from the previous step, and a loss at each epoch can be calculated. The training data loss and accuracy can then be calculated to determine the overall efficiency. The variables can be updated to predict stress levels via an optimized algorithm using a variety of algorithms such as a Gradient Descent algorithm. These steps can then be repeated for all the stress epoch counts to arrive at a trained machine learning model.

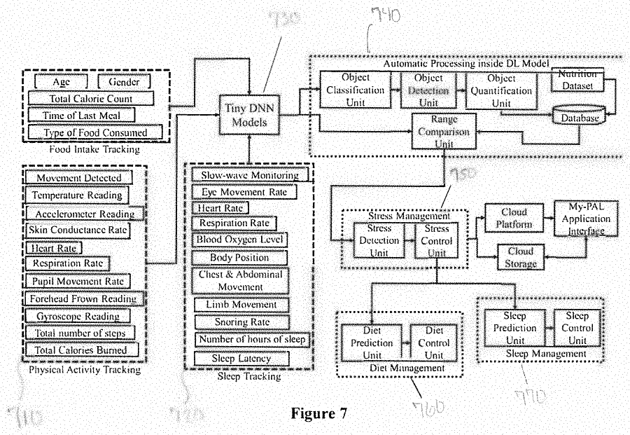

Referring to FIG. 7, an example data flow from the user device (My-PAL) to the user interface is represented. In some examples, data (e.g., physical activity data 710, food intake data, and/or sleep data 720) can be collected via the user device 100 and the characteristics for use in analyzing the stress behavior of the person can be fed into the deep machine learning models 730. The deep machine learning models 730 may include automatic processing unit 740 to perform automatic processing and range comparison. For example, the automatic processing unit 740 can include an object classification unit to identify objects from the input data and/or features. The object classification unit can pass the data to an object detection unit using the input data along with the object classification to identify objects from the data. The resulting object detection unit output can pass to an object quantification unit to identify a quantity of the object in the input data. A nutritional dataset can be used with stored data to determining a calorie count by applying the nutritional information to the detected objects and object counts. The information can then be stored.

The stress management unit 750 may include stress detection and control units. The stress detection unit can use data from the neural network models or contain addition machine learning models to detect the stress level based on the data. The output of the stress detection unit can pass as an input of the stress control unit, where recommendations can be provided based on the data and the detected stress level of the user. The control unit may be connected to a diet management unit 760 and sleep management unit 770, for example, which may facilitate improvement in the lifestyle of the user. Each of these units may take the data from the sensors and/or features extracted from the data along with the detected stress level and provide additional predictions and recommendations for presentation to the user on the user interface. Also shown in FIG. 7 is the ability to send the information to a cloud platform. The cloud platform can be used to store the information, execute any portions of the models, and/or provide the interface to the user at a location separate from the user device 100 (e.g., remote access and monitoring).

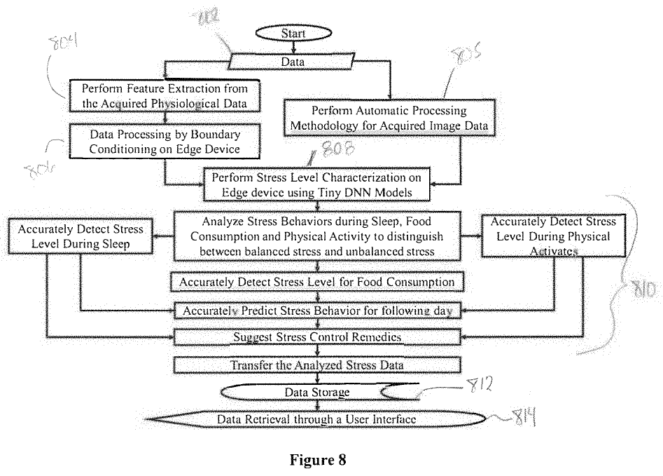

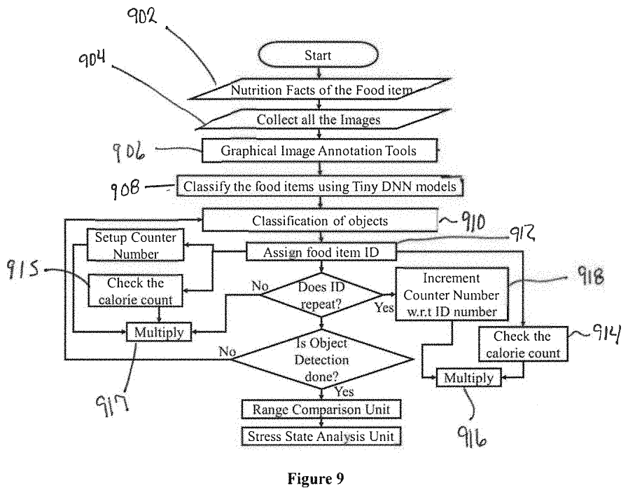

Referring to FIG. 8, an embodiment of stress behavior analysis, utilizing data obtained from the user device 100 and provided to the user via the user interface is represented. The data from the user device 100, for example, the IoT component 110 can be captured (step 802) and features can be extracted (step 804), different for both visual (step 805) and non-visual data (step 806). Feature extraction can comprise any of the feature extraction techniques as described herein. Data pre-processing (step 806) can be performed for the extracted data. Additionally or alternatively, data from the user device 100 may be subjected to automatic processing (step 805), as will be disclosed herein with respect to FIG. 9. The data and/or features can then be fed to the tiny DNN models (step 808) for stress behavior analyses, as described herein. The output of step 808 can be a detected stress level.

Stress prediction can be performed and control measures are implemented accordingly (steps 810). Once detected, the stress levels can be analyzed during the various activities throughout the day. For example the stress behaviors during sleep, food consumption, and physical activity can be analyzed to distinguished between balanced stress and unbalanced stress. For example, the stress levels can be individually determined by the neural network models to determine if the stress level is consistent between the various activities. In some aspects, a specific stress detection step can be carried out to determine the stress level during each activity (e.g., sleep, physical activity, and food consumption) to more accurately provide feedback. The resulting stress detection levels by activity can then be fed to a prediction model to predict stress behaviors for the following day. The stress prediction can be based on a machine learning model based on past behaviour, or correlations across a variety of users can be used in the prediction step. The predicted stress level(s) throughout the next day can then be used as inputs to provide suggestions and stress control remedies. The resulting output data set (e.g., the detected stress levels, predicted stress levels, and suggestions and remedies) can the be transferred for further processing and storage. The results of the analysis can be stored (step 812) and/or provided to a user (step 814), such as via a user interface.

Referring to FIG. 9, an example of a process for automatically processing data from a user device such as the user device 100 (My-PAL) of FIG. 1, for example, for image detection, object classification, and/or calorie quantification is shown. For example, in the embodiment of FIG. 9, information, such as nutrition facts, may be associated with various food items (step 902). The nutritional information may be stored in a database, which can be updated when information is needed and/or as nutritional information on new food items is available. Images of food items consumer by a user may be collected, for example, via an image capture device (e.g., a camera) associated with the user device 100 (step 904). Data and/or features may be extracted from the collected images, for example, to normalize or format the images for input into the classification models. The resulting processed images can then be fed to the machine learning models (e.g., DNN models) to perform classification of the objects within the images to classify the food items (steps 908 and 910) and identify and notate the food(s) present within the images (step 906), which are consumed by the user. Additional information, for example, portion size may also be extracted and notated. The analyzed results may be fed to the range comparison unit. The sensor data without images may also be fed into the range comparison unit.

In some embodiments, the detected, classified and labeled images may be assigned with a unique identification number (step 912) for correlation with the nutritional information. Nutritional information regarding the food items being detected may be determined and stored in a database, which can be accessed (steps 914 and 915). Based on the repetition of the objects in the plate, the identification number may be iterated (steps 916 and 917). The calorie information of the consumed food may be derived based on the identification number. If the identification number is repeated, the nutrition information may also be iterated with respect to the identification number (step 918). Thus, the quantities of foods consumed can be processed without any manual inputs.

An exemplary algorithm for implementing the method for automatic processing under the assumption of a person eating a donut and a bagel in a meal can be shown in the following algorithm:

| | |
|---|---|
| 1. | Initialize variables D1 and B1 to zero. |
| 2. | Obtain the detected, classified object and assign |
| | it to a variable or ID number D1 |
| | (assumption that the object is a donut). |
| 3. | Assign value count of D1, vD1 to one. |
| 4. | Retrieve the nutrition fact information stored with |
| | ID D1 from database. Therefore, calories |
| | associated with that ID, D1 (cD1) are obtained. |
| 5. | Continue to the next object that is classified. |
| 6. | If object is already assigned to ID then: |
| 7. | iterate the variable vD1 one time; |
| 8. | multiply vD1 with calorie information i.e., cD1 |
| 9. | Else |
| 10. | Assign new ID for the detected classified object B1 |
| | (assumption that the new object is a bagel). |
| 11. | Repeat step 4 with variable B1. |
| 12. | End if |
| 13. | Repeat the above steps for all the classified images. |
| 14. | Update the total calculated calorie (tc) count for each |
| | individual ID along with the time of |
| | consumption in the database. |

An example of process of automatic calorie quantification in My-PAL is explained in the following algorithm:

| | |
|---|---|
| 1. | Collect mages for testing and training the model. |
| 2. | Convert formats of the images from JPEG to |
| | XML after creating bounding boxes using a |
| | graphical image annotation tool. |
| 3. | Create multiple bounding boxes in various |
| | images for the same feature, called priors, using |
| | annotation tool. |
| 4. | Using box-coder, make the dimensions of the |
| | priors equal. |
| 5. | By considering the concept of IOU (Intersection |
| | Over Union), the matched and unmatched |
| | thresholds for matching the ground truth boxes |
| | to priors are set. In some examples, this |
| | may be mandatory as the model may not be |
| | ready for training if the match has not been |
| | made. |
| 6. | Images in XML format are made equal in size |
| | either by using reshape or resize functions. |
| 7. | Using convolution and rectified linear functions, |
| | the feature maps are assigned to every |
| | image sent to the model. |
| 8. | Based on these features, the images are either |
| | sent to regression or classification where the |
| | objects are detected through boxes in the images. |

Repeat the above steps for each of the images.

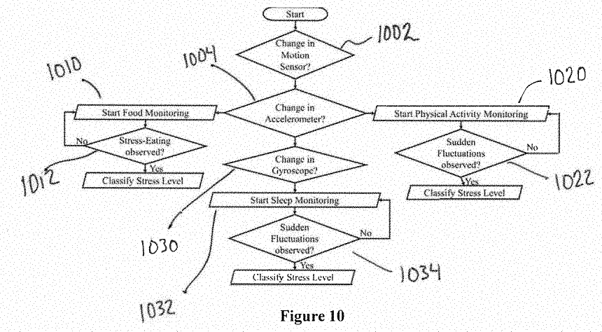

An example methodology for automatic stress detection, for example, based upon or during sleep and physical activity, is shown in FIG. 10. In this example, a movement sensor such as a motion sensor may be always on, and when there is a change detected (step 1002), the movement sensor (e.g., an accelerometer, etc.) may be checked (step 1004) for a change in position of the user to track the physical movement. The system may begin monitoring food consumption (step 1010) and physical activity monitoring (step 1020) to detect stress level fluctuations, for example, whether stress-eating is detected (step 1012) or fluctuations in activity are detected (step 1022) as the physical movement could be caused by either activity. In the process, depending upon gyroscope and position of the user (step 1030), the sleep activity may be monitored (step 1032). If the process continues, stress level fluctuations during sleep may be monitored (step 1034) and stress level predictions based on these levels may be made. This method demonstrates that each type of stress level monitoring can be detected throughout the day, where the assignment of the stress level can be based on the sensor inputs detected from the user. As the type of activity changes, the stress level detection models and types of data used in the models can change.

In some embodiments, the stress behavior of the person during sleep and physical activity may be performed periodically, such as performed about every 15 minutes, making it available in near real time or real time (e.g., substantially instantaneous). The detected stress may be considered as the input for the following day stress predictions, allowing the user to understand and act on maintaining stress. The following algorithm demonstrates one such detection method.

| | |
|---|---|
| 1. | Declare string variables brain wave voltage BWV, |
| | frequency - cycles per second CPS |
| | and Stress Level SL. |
| 2. | while time t1 not = 0 do |
| 3. | Update t1 at instance 1. |
| 4. | Start monitoring and gathering physiological signal |
| | data. |
| 5. | if BWV == ’low’ && t1 == 30 && 12>CPS<14 |
| 6. | then |
| 7. | Start t2 time at instance 1; user drifting to sleep. |
| 8. | else if t2 != to 0 && BWV == ’high’ && CPS<4 |
| 9. | then |
| 10. | Update t2 to current value at instance 2 ; user in |
| | deep sleep. |
| 11. | else if t2 != to 0 && BWV == ’high’ && EM 6= 0 |
| 12. | then |
| 13. | Update t2 at instance 3; user is in REM stage. |
| 14. | else |
| 15. | user is in light sleep. |
| 16. | end if |
| 17. | if t2 != to 0 && BWV == ’low’ && CPS > 13 then |
| 18. | Start t3 at instance 1; user woke up at alert state. |
| 19. | else |
| 20. | Start t3 timer; user woke up relaxed. |
| 21. | end if |
| 22. | if t1 == 0 then |
| 23. | Stop monitoring and gathering physiological |
| | signal data. |
| 24. | else |
| 25. | Repeat steps from 6 through 20. |
| 26. | end if |
| 27. | end while |
| 28. | Compare the gathered data from every instance to the |
| | characterization to detect SL of the |
| | user. |
| 29. | Repeat the steps from 5 through 28. |

In some embodiments, the stress behavior of the person during sleep and regular physical activity may be performed according to fuzzy logic, as shown in the following algorithm:

| | |
|---|---|
| 1. | Initialize the input random variables to zero. |
| 2. | Initialize the output random variable Stress Level Detection |
| | SLD to zero. |
| 3. | Assign these input variables to the initialized variables |
| | respectively. |
| 4. | Compare the input data to the range of the parameters. |
| 5. | while the input data is in the range do |
| 6. | Assign value count of variables accordingly. |
| 7. | if variable is already assigned to a number then |
| 8. | Replace the existing variable with the latest input |
| 9. | else |
| | Continue to the next variable assignment. |
| | end if |
| 10. | Repeat the steps from 5 through 10 until all the |
| | variables are assigned. |
| 11. | end while |
| 12. | if any variable = 0 then |
| 13. | Repeat from step 4. |
| 14. | end if |
| 15. | Detect Stress State during sleep and physical activity. |

Predict Stress behavior for following day.

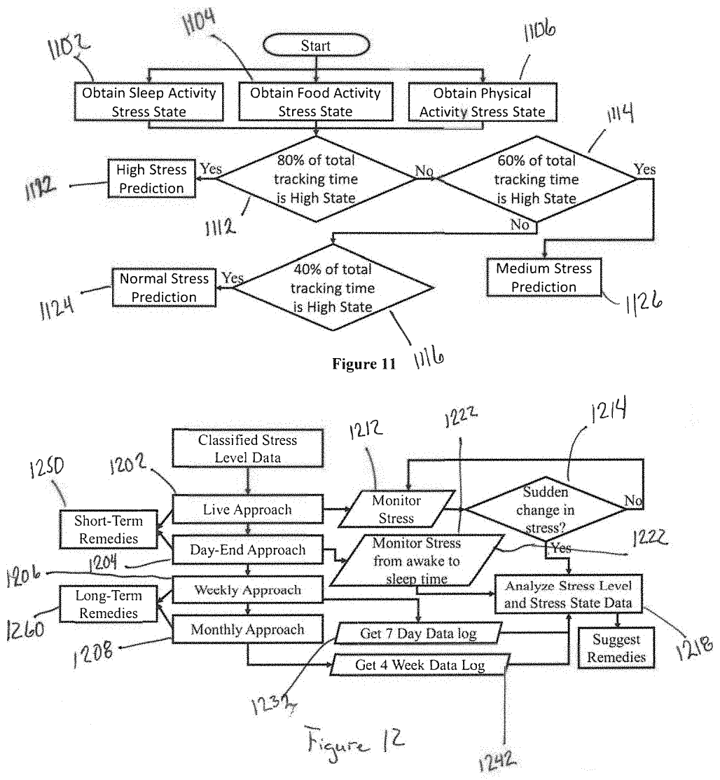

An example stress prediction method and system for My-PAL is discussed with respect to FIG. 11. In some embodiments, the stress prediction unit may work based on the instantaneous stress state detection unit. For example, the unit may enable the user to prepare for upcoming circumstances and provide a time period for the user to get back the control of his body and thoughts by taking certain stress control remedies provided by the system. For example, in the embodiment of FIG. 11, the method may include obtain a sleep activity stress state (step 1102), a food activity stress state (step 1104), and a physical activity stress state (step 1106). Based upon the amount (e.g., proportion and/or percentage) of time that is spent in a heightened or “High” stress state (steps 1112, 1114, and/or 1116), a prediction as to the user's stress may be determined (steps 1122, 1124, 1126). The total tracking time considered may be 24 hours, though any period of time may be suitable. The prediction analysis in My-PAL may consider factors such as sleep latency in minutes which is the length of time the user has taken to drift in to sleep, the quality of sleep and the total number of hours the user has actually slept. For proper, good quality sleep, in some examples the sleep latency should be in the range of about 10 to about 20 minutes. An example stress prediction method is presented in the following algorithm:

| | |
|---|---|
| 1. | Retrieve the updated variables t1, t2 and t3 at instances 1. |
| 2. | Declare and initialize the variable sleep latency as L to 0, |
| | actual time slept to ATS to 0, |
| | Stress Prediction to SP. |
| 3. | Calculate the difference between the variables t2 and t1 |
| | to update L and t3 and t2 to |
| | update ATS. |
| 4. | while SL is detected do |
| 5. | if 0.8*ATS == ’M’ OR 0.8*ATS == ’H’ OR 0.8*ATS |
| | == ‘MH’ && 35<L >45 |
| 6. | then |
| 7. | SP == ’MH’; user could experience rapid mood |
| | swings, irritability, sleeplessness |
| | and fatigue during day. |
| 8. | else if 0.6*ATS == ’M’ OR 0.6*ATS == ’L’ OR |
| | 0.6*ATS == ’ML’ && 20<L>35 |
| 9. | then |
| 10. | SP == ’ML’; user could experience some mood |
| | swings and tiredness during day. |
| 11. | else if 0.8*ATS == ’L’ && 10<L>20 |
| 12. | then |
| 13. | SP == ’L/N’; user could remain active and |
| | happy for the course of the day. |
| 14. | end if |
| 15. | end while. |
| 16. | Repeat the steps from 5 through 15 to predict stress |
| | every time. |

In various embodiments, stress control may be divided into long term and short term to take advantage of control mechanisms for each, which may include at least four different approaches as shown in FIG. 12, for example, a live approach 1202, a day-end approach 1204, a weekly approach 1206, and a monthly approach 1208. For example, the live approach 1202 may monitor stress (step 1212) for sudden changes (step 1214). The day-end approach 1204 may monitor stress during waking hours (step 1222). The weekly approach 1206 may establish a 7-day log of stress levels (step 1232). The monthly approach 1208 may establish a 4-week log of stress levels (step 1242). The control remedies in the various datasets may be compared to the classified stress level data from a user device, for example, to establish one or more remedies (step 1218). The live approach 1202 and day-end approach 1204 may be considered short-term as the duration of a person during stress may be relatively low when compared to prolonged exposure to stress. For example, the live approach 1202 and day-end approach 1204 may be used to implement short-term remedies 1250; additionally, the weekly approach 1206 and monthly approach 1208 may be used to implement short-term remedies 1260.

In some embodiments, the live approach 1202 and the day-end approach 1204 can be categorized under acute or stage 1 chronic stress, which are not severe when compared to chronic stress stages 2 and 3. The weekly approach 1206 and monthly approach 1208 may be considered long-term, as the time duration of the stress is higher. Such prolonged exposures can cause health issues for the users. These approaches can be categorized under stages 2 and 3 of chronic stress, anxiety issues, post-traumatic stress, and may be important to stay in control of for a person to live happily and stay healthy.

An example method for automatic stress control advisory is shown in the following algorithm:

| | |
|---|---|
| 1. | Initialize the input random variables l, d, w and m to zero. |
| 2. | Initialize the output random variables st and lt to zero. |
| 3. | Assign the input variables to live, day-end, weekly and monthly, respectively. |
| 4. | Assign output variables to short-term and long-term respectively. |
| 5. | Assign classified stress level data to D with L, M and H as Low, Medium and High |
| | levels. |
| 6. | Declare a Boolean variable ‘data’ to ‘true’ for D from wearables. |
| 7. | while data==true do |
| 8. | Move the contents of D to l. |
| 9. | while l==L \| \| l==M \| \| l==H do |
| 10. | Display Short-Term Remedies for any stress levels, L, M and H. |
| 11. | while l !=0 do |
| 12. | Move the contents of l to d. |
| 13. | if 0.08*l == L then |
| 14. | Display Short-Term Remedies for low stress. |
| 15. | else if 0.08*l == M then |
| 16. | Display Short-Term Remedies for medium stress. |
| 17. | else if 0.08*l == H then |
| 18. | Display Short-Term Remedies for High stress. |
| 19. | end if |
| 20. | while d !=0 do |
| 21. | Move the contents of d to w. |
| 22. | if 2*d >= H then |
| 23. | Display Long-Term Remedies for low stress. |
| 24. | else if 4*d >= H then |
| 25. | Display Long-Term Remedies for medium |
| | stress. |
| 26. | else if 5*d >= H then |
| 27. | Display Long-Term Remedies for High |
| | stress. |
| 28. | end if |
| 29. | while w !=0 do |
| 30. | Move the contents of w to m. |
| 31. | if l*w >= H then |
| 32. | Display Long-Term Remedies for low stress. |
| 33. | else if 2*w >= H then |
| 34. | Display Long-Term Remedies for medium |
| | stress. |
| 35. | else if 3 *w >= H then |
| 36. | Display Long-Term Remedies for |
| | High |
| | stress. |
| 37. | end if |

Example remedies for the short-term approaches are listed in the Table 1. Example remedies for the Long-term approaches are listed in the Table 2 showing Week and Month Remedies.

TABLE 1
Live and Day approach Remedies.

| Low Stress | Medium Stress | High Stress |
|---|---|---|
| (Alarm Reaction) | (Resistance) | (Exhaustion) |
| 1 min meditation | 3 min meditation | 15 min meditation |
| 1 min heavy breathing | 3 min heavy breathing | 15 min heavy breathing |
| 30 sec smiles | 1 min smiles | 10 heavy laughter’s |
| Scroll through photos | Read motivational quotes | Watch funny videos |
| Look at diagrams | Read a book | Write how you feel |

TABLE 2
Week and Month approach Remedies.

| Low Stress | Medium Stress | High Stress |
|---|---|---|
| (Alarm Reaction) | (Resistance) | (Exhaustion) |
| 15 min meditation | Automatic display of | Customized Workout |
| | paintings | |
| 15 min heavy breathing | Take a walk | Customized meal plan |
| 1 min smile | 10 min Laughter | Display Therapy dog’s |
| | | location |
| Scroll through photos | Automatic display of | Play music |
| | photos | |
| Do Yoga | Automatic Yoga classes | Display Workout |
| | | Instructor’s classes |
| Take a bath | Automatic Karaoke | Automatic ambience |
| | | control |
| Practice Painting | Open online painting | Suggest Nearby painting |
| | applications | sessions |
| Stay warm | Regulate room | Customize sleep plan |
| | temperature | |

The systems and method described herein provide a number of advantages with regard to food classification, consumption, monitoring, and stress control. For example, the present systems and methods allow for users to be educated regarding the relationship between stress, stress-eating, and normal eating using the stress detection and feedback mechanisms. The system also allows for automatic classification and quantification of food items. The variations in stress level with regard to food consumption habits can also be analyzed and presented to the user. The system also allows the ability to provide techniques to control variations in stress levels of the user through the prediction and remediation recommendations provided to the user. The system also allows a wearable device that monitors the intake of food while taking leftovers or unconsumed food into account. Overall, the present system is a fully automated system that allows for tracking of sleep, activity, and food consumption while providing an automatically determined stress level with associated feedback and recommendations.

Having described various devices, systems, and methods, certain aspects can include, but are not limited to:

In a first aspect, a wearable device comprises: multiple sensors; a memory; a processor coupled to the memory and the multiple processors; and executable code stored in the memory that when executed by the processor, causes the processor to: receive data from at least some of the multiple sensors, the data indicating physical activity of a wearer of the wearable device, food consumption of the wearer, and sleeping habits of the wearer; and determine an estimated stress level of the wearer based on the physical activity of the wearer, the food consumption of the wearer, and the sleeping habits of the wearer.

A second aspect can include the wearable device of the first aspect, wherein executing the executable code further causes the processor to recommend a stress control remedy to the wearer.

A third aspect can include the wearable device of the second aspect, wherein the stress control remedy includes exercise plans.

A fourth aspect can include the wearable device of the second or third aspect, wherein the stress control remedy includes a sleep management plan.

A fifth aspect can include the wearable device of any one of the second to fourth aspects, wherein the stress control remedy including the sleep management plan includes modifying an ambiance of a sleeping location, modifying a temperature of the sleeping location, modifying a posture of the wearer by controlling a controllable bed, modifying a mattress temperature, controlling an aromatic dispenser in the sleeping location, or playing of audio in the sleeping location.

A sixth aspect can include the wearable device of any one of the second to fifth aspects, wherein the stress control remedy includes recommendations for two or more of sleep management, food consumption, or exercise.

A seventh aspect can include the wearable device of any one of the second to sixth aspects, wherein the stress control remedy is one of a short-term remedy or a long-term remedy.

An eighth aspect can include the wearable device of any one of the second to seventh aspects, wherein the stress control remedy is determined according to location services of the wearable device that determine and provide a physical location of the wearer.

A ninth aspect can include the wearable device of any one of the second to eighth aspects, wherein the stress control remedy is a food consumption plan.

A tenth aspect can include the wearable device of any one of the first to ninth aspects, wherein executing the executable code further causes the processor to automatically log food consumed by the wearer based on the data from at least some of the multiple sensors without user input.

An eleventh aspect can include the wearable device of the tenth aspect, wherein the data from at least some of the multiple sensors is images of the food consumed by the wearer.

A twelfth aspect can include the wearable device of the tenth or eleventh aspect, wherein executing the executable code further causes the processor to automatically quantify caloric intake for the food consumed by the wearer based on the data from at least some of the multiple sensors without user input.

A thirteenth aspect can include the wearable device of any one of the first to twelfth aspects, wherein executing the executable code further causes the processor to determine and consider leftover food and unconsumed calories in determining the estimated stress level of the wearer based on the food consumption of the wearer.

A fourteenth aspect can include the wearable device of any one of the first to thirteenth aspects, wherein determining the estimated stress level of the wearer based on the physical activity of the wearer, the food consumption of the wearer, and the sleeping habits of the wearer determines fluctuations in stress throughout a day.

A fifteenth aspect can include the wearable device of any one of the first to fourteenth aspects, wherein determining the estimated stress level of the wearer based on the physical activity of the wearer, the food consumption of the wearer, and the sleeping habits of the wearer creates a correlation and relationship between eating habits of the wearer, physical activity of the wearer, sleep of the wearer, and stress of the wearer.

A sixteenth aspect can include the wearable device of any one of the first to fifteenth aspects, wherein the processor is one of a hardware accelerated machine learning processor or an artificial intelligence processor.

A seventeenth aspect can include the wearable device of any one of the first to sixteenth aspects, wherein the processor is configured to communicate with an Internet-of-Things device to regulate stress of the wearer based at least partially on the determined estimated stress level of the wearer.

In an eighteenth aspect, a method for stress control comprises: capturing or receiving data from at least some of the multiple sensors, the data indicating physical activity of a wearer of the wearable device, food consumption of the wearer, and sleeping habits of the wearer; determining an estimated stress level of the wearer based on the physical activity of the wearer, the food consumption of the wearer, and the sleeping habits of the wearer; and automatically determining and recommending a stress control remedy to the wearer based on the determined estimated stress level and at least one of the physical activity of the wearer, the food consumption of the wearer, or the sleeping habits of the wearer.

A nineteenth aspect can include the method of the eighteenth aspect, wherein the stress control remedy creates a normalized stress pattern for the wearer.

A twentieth aspect can include the method of the eighteenth or nineteenth aspect, wherein the stress control remedy includes exercise plans.

A twenty first aspect can include the method of any one of the eighteenth to twentieth aspects, wherein the stress control remedy includes a sleep management plan.

A twenty second aspect can include the method of any one of the eighteenth to twenty first aspects, wherein the stress control remedy includes a food consumption plan.

A twenty third aspect can include the method of any one of the eighteenth to twenty second aspects, wherein the stress control remedy is one of short-term or long-term.

A twenty fourth aspect can include the method of any one of the eighteenth to twenty third aspects, further comprising determining a physical location of the wearer, and wherein the stress control remedy is determined at least partially according to the physical location of the wearer.

A twenty fifth aspect can include the method of any one of the eighteenth to twenty fourth aspects, further comprising automatically classifying and logging food consumed by the wearer without user input, based at least partially on images captured of the food consumed by the wearer.

A twenty sixth aspect can include the method of the twenty fifth aspect, further comprising automatically determining a caloric quantification of the food consumed by the wearer without user input, based at least partially on the images captured of the food consumed by the wearer.

A twenty seventh aspect can include the method of the twenty sixth aspect, further comprising determining the estimated stress level of the wearer based at least partially on the caloric quantification of the food consumed by the wearer.

A twenty eighth aspect can include the method of any one of the eighteenth to twenty seventh aspects, further comprising automatically determining leftover food left unconsumed by the wearer without user input, based at least partially on the images captured of the leftover food, and determining a caloric quantification of the leftover food.

A twenty ninth aspect can include the method of the twenty eighth aspect, further comprising making a stress control recommendation to the wearer based at least partially on the leftover food.

A thirtieth aspect can include the method of any one of the eighteenth to twenty ninth aspects, further comprising determining the estimated stress level of the wearer based at least partially on physiological parameter changes during sleep of the wearer.

A thirty first aspect can include the method of any one of the eighteenth to thirtieth aspects, further comprising automatically monitoring the daily activities of the wearer to determine stress fluctuations for the wearer throughout a day.

A thirty second aspect can include the method of any one of the eighteenth to thirty first aspects, further comprising determining the estimated stress level of the wearer based at least partially on workouts by the wearer, a number of steps taken by the wearer, or a number of calories burned by the wearer.

A thirty third aspect can include the method of any one of the eighteenth to thirty second aspects, further comprising making a diet recommendation to the wearer based on food consumed by the wearer, leftover food left unconsumed by the wearer, or a daily caloric budget of the wearer to control stress of the wearer.

A thirty fourth aspect can include the method of any one of the eighteenth to thirty third aspects, wherein determining the estimated stress level of the wearer based on the physical activity of the wearer, the food consumption of the wearer, and the sleeping habits of the wearer creates a correlation and relationship between eating habits of the wearer, physical activity of the wearer, sleep of the wearer, and stress of the wearer.

A thirty fifth aspect can include the method of any one of the eighteenth to thirty fourth aspects, further comprising providing the recommended stress control remedy to the wearer via a graphical user interface of an application presented on a smart device, a computer, or a web interface.

A thirty sixth aspect can include the method of the thirty fifth aspect, further comprising providing information about the physical activity of the wearer, the food consumption of the wearer, the sleeping habits of the wearer, or the estimated stress level of the wearer via the graphical user interface.

A thirty seventh aspect can include the method of any one of the eighteenth to thirty sixth aspects, further comprising determining future stress predictions.

A thirty eighth aspect can include the method of the thirty seventh aspect, further comprising monitoring physiological data of the wearer including sleep latency, and the food consumption to determine stress predictions for a following day.

A thirty ninth aspect can include the method of any one of the eighteenth to thirty eighth aspects, implemented by an Internet-of-Medical-Things (IoMT) device.

A fortieth aspect can include the method of the thirty ninth aspect, wherein the IoMT device is implemented in a healthcare Cyber-Physical System (H-CPS) framework.

A forty first aspect can include the method of the fortieth aspect, wherein the H-CPS framework includes at least four approached for stress control.

A forty second aspect can include the method of the fortieth or forty first aspect, wherein the H-CPS framework provides unified and interrelated diet management, sleep management, and stress management.

Embodiments are discussed herein with reference to the Figures. However, those skilled in the art will readily appreciate that the detailed description given herein with respect to these figures is for explanatory purposes as the systems and methods extend beyond these limited embodiments. For example, it should be appreciated that those skilled in the art will, in light of the teachings of the present description, recognize a multiplicity of alternate and suitable approaches, depending upon the needs of the particular application, to implement the functionality of any given detail described herein, beyond the particular implementation choices in the following embodiments described and shown. That is, there are numerous modifications and variations that are too numerous to be listed but that all fit within the scope of the present description. Also, singular words should be read as plural and vice versa and masculine as feminine and vice versa, where appropriate, and alternative embodiments do not necessarily imply that the two are mutually exclusive.

It is to be further understood that the present description is not limited to the particular methodology, compounds, materials, manufacturing techniques, uses, and applications, described herein, as these may vary. It is also to be understood that the terminology used herein is used for the purpose of describing particular embodiments only, and is not intended to limit the scope of the present systems and methods. It must be noted that as used herein and in the appended claims (in this application, or any derived applications thereof), the singular forms “a,” “an,” and “the” include the plural reference unless the context clearly dictates otherwise. Thus, for example, a reference to “an element” is a reference to one or more elements and includes equivalents thereof known to those skilled in the art. All conjunctions used are to be understood in the most inclusive sense possible. Thus, the word “or” should be understood as having the definition of a logical “or” rather than that of a logical “exclusive or” unless the context clearly necessitates otherwise. Structures described herein are to be understood also to refer to functional equivalents of such structures. Language that may be construed to express approximation should be so understood unless the context clearly dictates otherwise.

Unless defined otherwise, all technical and scientific terms used herein have the same meanings as commonly understood by one of ordinary skill in the art to which this description belongs. Preferred methods, techniques, devices, and materials are described, although any methods, techniques, devices, or materials similar or equivalent to those described herein may be used in the practice or testing of the present systems and methods. Structures described herein are to be understood also to refer to functional equivalents of such structures. The present systems and methods will now be described in detail with reference to embodiments thereof as illustrated in the accompanying drawings.

From reading the present disclosure, other variations and modifications will be apparent to persons skilled in the art. Such variations and modifications may involve equivalent and other features which are already known in the art, and which may be used instead of or in addition to features already described herein.

Although Claims may be formulated in this Application or of any further Application derived therefrom, to particular combinations of features, it should be understood that the scope of the disclosure also includes any novel feature or any novel combination of features disclosed herein either explicitly or implicitly or any generalization thereof, whether or not it relates to the same systems or methods as presently claimed in any Claim and whether or not it mitigates any or all of the same technical problems as do the present systems and methods.

Features which are described in the context of separate embodiments may also be provided in combination in a single embodiment. Conversely, various features which are, for brevity, described in the context of a single embodiment, may also be provided separately or in any suitable sub-combination. The Applicants hereby give notice that new claims may be formulated to such features and/or combinations of such features during the prosecution of the present Application or of any further Application derived therefrom.

What is claimed is:

1. A wearable device, comprising:
a plurality of sensors;
a memory;
a processor coupled to the memory and the plurality of sensors; and
executable code stored in the memory that when executed by the processor, causes the processor to:
receive data from at least some of the plurality of sensors, the data indicating physical activity of a wearer of the wearable device, food consumption of the wearer, and sleeping habits of the wearer; and
determine an estimated stress level of the wearer based on the physical activity of the wearer, the food consumption of the wearer, and the sleeping habits of the wearer.

2. The wearable device of claim 1, wherein executing the executable code further causes the processor to recommend a stress control remedy to the wearer.

3. The wearable device of claim 2, wherein the stress control remedy includes exercise plans, a sleep management plan, a food consumption plan, or combinations thereof.

4. The wearable device of claim 3, wherein the stress control remedy including the sleep management plan includes modifying an ambiance of a sleeping location, modifying a temperature of the sleeping location, modifying a posture of the wearer by controlling a controllable bed, modifying a mattress temperature, controlling an aromatic dispenser in the sleeping location, or playing of audio in the sleeping location.

5. The wearable device of claim 2, wherein the stress control remedy includes recommendations for two or more of sleep management, food consumption, or exercise.

6. The wearable device of claim 1, wherein executing the executable code further causes the processor to automatically log food consumed by the wearer based on the data from at least some of the multiple sensors without user input, wherein the data from at least some of the multiple sensors is images of the food consumed by the wearer.

7. The wearable device of claim 6, wherein executing the executable code further causes the processor to automatically quantify caloric intake for the food consumed by the wearer based on the data from at least some of the multiple sensors without user input.

8. The wearable device of claim 1, wherein executing the executable code further causes the processor to determine and consider leftover food and unconsumed calories in determining the estimated stress level of the wearer based on the food consumption of the wearer.

9. The wearable device of claim 1, wherein determining the estimated stress level of the wearer based on the physical activity of the wearer, the food consumption of the wearer, and the sleeping habits of the wearer creates a correlation and relationship between eating habits of the wearer, physical activity of the wearer, sleep of the wearer, and stress of the wearer.

10. The wearable device of claim 1, wherein the processor is one of a hardware accelerated machine learning processor or an artificial intelligence processor.

11. The wearable device of claim 1, wherein the processor is configured to communicate with an Internet-of-Things device to regulate stress of the wearer based at least partially on the determined estimated stress level of the wearer.

12. A method for stress control, comprising:
capturing or receiving data from at least some of the multiple sensors, the data indicating physical activity of a wearer of the wearable device, food consumption of the wearer, and sleeping habits of the wearer;
determining an estimated stress level of the wearer based on the physical activity of the wearer, the food consumption of the wearer, and the sleeping habits of the wearer; and
automatically determining and recommending a stress control remedy to the wearer based on the determined estimated stress level and at least one of the physical activity of the wearer, the food consumption of the wearer, or the sleeping habits of the wearer.

13. The method of claim 12, wherein the stress control remedy creates a normalized stress pattern for the wearer.

14. The method of claim 12, further comprising automatically classifying and logging food consumed by the wearer without user input, based at least partially on images captured of the food consumed by the wearer.

15. The method of claim 14, further comprising:
automatically determining a caloric quantification of the food consumed by the wearer without user input, based at least partially on the images captured of the food consumed by the wearer; and
determining the estimated stress level of the wearer based at least partially on the caloric quantification of the food consumed by the wearer.

16. The method of claim 12, further comprising:
automatically determining leftover food left unconsumed by the wearer without user input, based at least partially on the images captured of the leftover food, and determining a caloric quantification of the leftover food; and
making a stress control recommendation to the wearer based at least partially on the leftover food.

17. The method of claim 12, further comprising determining the estimated stress level of the wearer based at least partially on workouts by the wearer, a number of steps taken by the wearer, or a number of calories burned by the wearer.

18. The method of claim 12, further comprising making a diet recommendation to the wearer based on food consumed by the wearer, leftover food left unconsumed by the wearer, or a daily caloric budget of the wearer to control stress of the wearer.

19. The method of claim 12, further comprising determining future stress predictions.

20. The method of claim 12, implemented by an Internet-of-Medical-Things (IoMT) device implemented in a healthcare Cyber-Physical System (H-CPS) framework.
